(19) United States
(12) Patent Application Publication
(10) Pub. No.: US 2015/0246199 A1
(43) Pub. Date: Sep. 3, 2015

(54) ARTICULATING FULL FACE MASK

(71) Applicant: KONINKLIJKE PHILIPS N.V., Eindhoven (NL)

(72) Inventors: Jerome Matula, JR., Apollo, PA (US); Gregory John Jablonski, Butler, PA (US); Antonio Bugamelli, Mars, PA (US); Derrick Blake Andrews, Markleton, PA (US)

(21) Appl. No.: 14/429,128

(22) PCT Filed: Aug. 27, 2013
(86) PCT No.: PCT/IB2013/058013

§ 371 Date: Mar. 18, 2015

Related U.S. Application Data
(60) Provisional application No. 61/702,843, filed on Sep. 19, 2012.

(51) Int. Cl. A61M 16/06; A61M 16/08

(52) U.S. Cl. CPC A61M 16/0622; A61M 16/0666; A61M 16/0683; A61M 16/08

(57) ABSTRACT

A cushion for use in a patient interface device includes a first portion adapted to sealingly engage about the nares of a patient, a second portion adapted to sealingly engage about the mouth of the patient, and a third portion configured to allow for the relative positioning of the first portion and the second portion to be selectively adjusted.

### CROSS-REFERENCE TO RELATED APPLICATIONS

This patent application claims the priority benefit under 35 U.S.C. §119(e) of U.S. Provisional Application No. 61/702,843 filed on Sep. 19, 2012, the contents of which are herein incorporated by reference.

### BACKGROUND OF THE INVENTION

1. Field of the Invention

The present invention generally relates to patient interfaces, and, more particularly, to full face masks for use in pressure support systems that supply a flow of gas to the airway of a patient.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation (NIV). It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), chronic obstructive pulmonary disease (COPD), or congestive heart failure (CHF).

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient.

Typically, patient interface devices include a mask shell or frame having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because such masks are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. It is also important that the interface device provide a tight enough seal against a patient's face without discomfort. A problem arises in that in order for the mask to maintain a seal without any undue gas leaks around the periphery of the mask, the mask may be compressed against the patient's face.

Conventional CPAP masks that seal by compression commonly cause uncomfortable pressure points and do not adjust well to different anatomical facial features. The conventional concept of a compression seal is to generally displace tissue on the patient's face in order to achieve a uniform seal. Tissue displacement results in pressure points, skin markings (i.e., red marks), indentations, and overall prolonged discomfort. Conventional seals also commonly distort outward upon compressing and in doing so tend to pull or push on portions of a patient's face in a manner which is uncomfortable and can commonly lead to unwanted leaks.

Some sleep apnea masks are provided in the form of full face masks. Full face masks are generally one piece structures that provide a seal around the entire nasal and oral area of the patient. In encompassing such generally large area, such masks are particularly susceptible to fitment and/or leakage issues.

Accordingly, a need exists for a patient interface device that improves upon existing devices, for example, to maximize patient comfort while minimizing leak, during delivery of a positive airway pressure or flow of gas to the airway of the user. More particularly, a need exists for an improved full face mask.

### SUMMARY OF THE INVENTION

As one aspect of the present invention a cushion for use in a patient interface device is provided. The cushion comprises a first portion adapted to sealingly engage about the nares of a patient, a second portion adapted to sealingly engage about the mouth of the patient, and a third portion configured to allow for the relative positioning of the first portion and the second portion to be selectively adjusted.

The third portion of the cushion may be formed as a part of the second portion. The third portion of the cushion may be formed as a part of the first portion. The first, second and third portions of the cushion may be portions of a single integral member. The first and second portions of the cushion may be formed as separate members that have been coupled together. The third portion of the cushion may be disposed between the first and second portions of the cushion. The first portion of the cushion may comprise one of a nasal mask, a pillows mask, or a cradle mask. The third portion of the cushion may comprise a bellows-like structure. The first, second and third portions of the cushion may be separate members selectively coupled together to form the cushion.

As another aspect of the invention, a patient interface device having a cushion is provided. The cushion includes: a first portion adapted to sealingly engage about the nares of a patient and a second portion adapted to sealingly engage about the mouth of the patient. At least one of the first portion or the second portion of the cushion is movable from a first position in which the first portion and the second portion are disposed relative to each other and a second position in which the first portion and the second portion are disposed in a different relative position with respect to each other.

The patient interface device may further include an adjustment mechanism coupled to the first and second portions of the cushion, the adjustment mechanism being adapted to selectively fix the first portion and the second portion in at least one of the first position or the second position. The adjustment mechanism may comprise: a first frame member coupled to the first portion of the cushion, a second frame member coupled to the second portion of the cushion, and a mechanism engaged with both the first frame member and the second frame member, the mechanism being adapted to selectively fix the relative positioning of the first frame member and the second frame member with respect to each other. The mechanism may comprise one of a screw-like member or a cord member.

As yet another aspect of the invention, a method of fitting a patient interface device having a cushion including a first portion adapted to sealingly engage about the nares of a patient and a second portion adapted to sealingly engage about the mouth of the patient is provided. The method comprises: disposing the patient interface device on the head of the patient such that one of the first portion and the second portion is sealingly engaged about a corresponding one of the nares or mouth of the patient and the other of the first portion and the second portion is spaced a distance from the other one of the nares or mouth of the patient, and adjusting the relative positioning of the first portion and the second portion to bring the other one of the first portion and the second portion into sealing engagement about the other one of the nares or mouth of the patient.

Adjusting the relative positioning of the first portion and the second portion may comprise selectively fixing the relative positioning of the first and second portions with respect to each other.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are provided for the purpose of illustration and description only and are not intended as a definition of the limits of the invention.

### BRIEF DESCRIPTION OF THE DRAWINGS

FIG. 1 shows a partially schematic view of a system adapted to provide a regimen of respiratory therapy to a patient, and includes an isometric view of an example patient interface device in accordance with the principles of the present invention disposed on the head of a patient;

FIG. 2 is an isometric view of the patient side of the patient interface device of FIG. 1;

FIG. 3 is a side view of another example patient interface device in accordance with the principles of the present invention disposed on the face of a patient in a first position;

FIG. 4 is another side view of the patient interface device of FIG. 3 disposed on the face of a patient in a different second position;

FIG. 5 is a side view of yet another example patient interface device in accordance with the principles of the present invention disposed on the face of a patient in a first position;

FIG. 6 is another side view of the patient interface device of FIG. 5 disposed on the face of a patient in a different second position;

FIG. 7 is a side view of a further example patient interface device in accordance with the principles of the present invention disposed on the face of a patient;

FIG. 8 is an exploded side view of the patient interface device of FIG. 7;

FIG. 9 is an isometric view of yet a further example patient interface device in accordance with the principles of the present invention; and

FIG. 10 is an isometric view of the patient side of the patient interface device of FIG. 9.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality) and the singular form of “a”, “an”, and “the” include plural referents unless the context clearly indicates otherwise.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

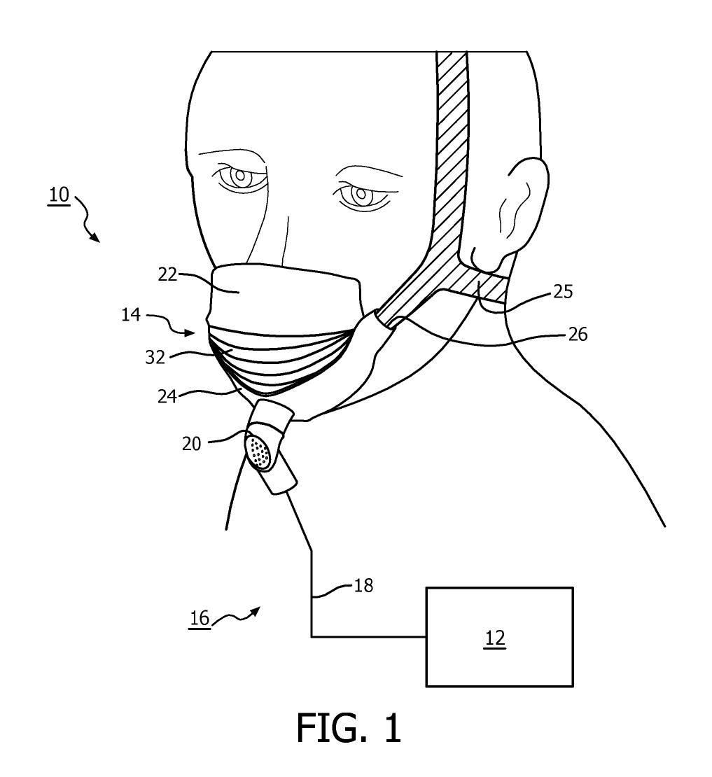

A system 10 adapted to provide a regimen of respiratory therapy to a patient is generally shown in FIG. 1 disposed on the head of a patient. System 10 includes a pressure generating device 12 (shown schematically), a patient interface device 14, and a patient circuit 16. Although system 10 is discussed as including pressure generating device 12, patient interface 14 and patient circuit 16, it is contemplated that other systems may be employed while remaining within the scope of the present invention.

Pressure generating device 12 is structured to generate a flow of breathing gas and may include, without limitation, ventilators, constant pressure support devices (such as a continuous positive airway pressure device, or CPAP device), variable pressure devices (e.g., BiPAp®, Bi-Flex®, or C-Flex™ devices manufactured and distributed by Philips Respironics of Murrysville, Pa.), and auto-titration pressure support devices.

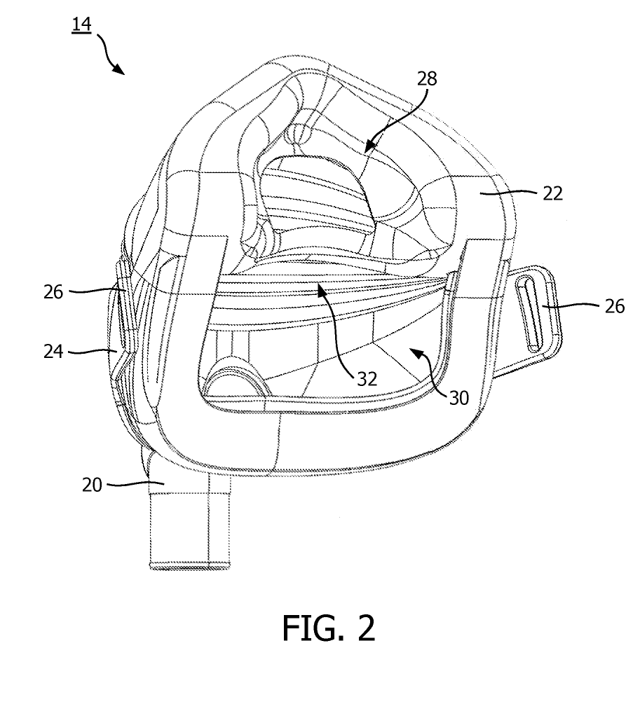

Patient circuit 16 is structured to communicate the flow of breathing gas from pressure generating device 12 to patient interface device 14. Typically, patient circuit 16 includes a conduit or tube which couples pressure generating device 12 and patient interface device 14. In the example embodiment illustrated in FIGS. 1 and 2, patient circuit 16 includes a conduit 18 (shown schematically) and an elbow 20 coupled to patient interface device 14.

Patient interface device 14 is a mask that, as shown in FIG. 1, is structured to be placed over the nasal and oral orifices of a patient. As shown in the example embodiment of FIGS. 1 and 2, patient interface device 14 includes a cushion 22 and a generally rigid frame 24 coupled thereto. Straps 25, such as shown in FIG. 1, may be attached to end portions 26 of frame 24 to secure patient interface device 14 to the patient's head. It is to be appreciated that embodiments of the present invention may be secured to the head of a patient using various types of headgear or mounting arrangements other than those illustrated and that embodiments of the present concept are not intended to be limited to any particular headgear or mounting arrangement. An opening in frame 24, to which elbow 20 is coupled, allows the flow of breathing gas from pressure generating device 12 to be communicated to interior spaces (not numbered) defined by cushion 22 and then to the airway of the patient.

The opening in frame 24 also allows the flow of exhalation gas (from the airway of such a patient) to be communicated to elbow 20 and an exhaust port assembly (not numbered) disposed therein. Although illustrated in separate elbow 20, it is contemplated that the exhaust port assembly may be incorporated into, for example and without limitation, patient interface 14 and/or different variations of patient circuit 16 without varying from the scope of the present invention.

Referring to FIG. 2, cushion 22 includes a first portion 28 adapted to sealingly engage about the nares of the patient. As shown in the illustrated embodiment, first portion 28 may be formed as a nasal mask, however, it is to be appreciated that first portion 28 may also be arranged in other forms (e.g., without limitation, a pillows mask, a cradle mask) without varying from the scope of the present invention. Continuing to refer to FIG. 2, cushion 22 also includes a second portion 30 adapted to sealingly engage about the mouth of the patient. Cushion 22 further includes a third portion 32 which is configured to allow for the relative positioning of first portion 28 and second portion 30 to be generally selectively adjusted so as to provide for optimum fitting of cushion 22, and thus patient interface device 14, to the face of the patient. By allowing relative motion between the nasal and oral portions (portions 28 and 30) of the mask, a better seal and improved comfort for the patient is achieved.

In the example embodiment illustrated in FIGS. 1 and 2, third portion 32 is formed as a part of second portion 30. More particularly, in the example embodiment illustrated in FIGS. 1 and 2, third portion 32 is formed as a spring-like bellows member that generally provides for the height of second portion 30 to be selectively varied, thus providing for the relative positioning of first portion 28 to be adjusted relative the second portion 30. Although shown as a spring-like bellows member in the embodiment illustrated in FIGS. 1 and 2, it is to be appreciated that third portion 32 may be formed as other suitable structures that allow for the height of second portion 30 to be similarly adjusted without varying from the scope of the present invention. In other embodiments, some examples of which are discussed below, third portion 32 may also be disposed between first portion 28 and second portion 30.

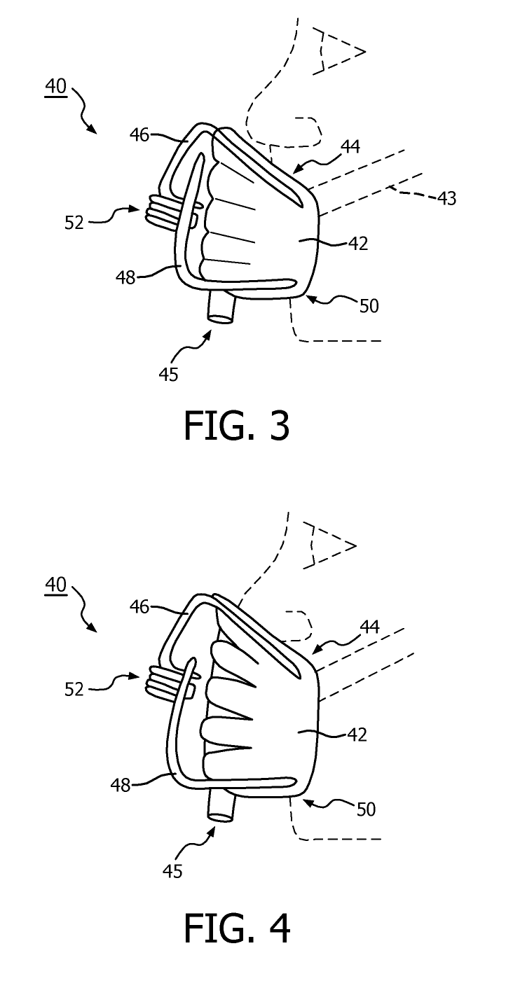

FIGS. 3 and 4 show side views of another example embodiment of a patient interface device 40 having a cushion 42 similar to cushion 22 previously discussed, disposed on the face of a patient (shown in dashed lines). More particularly, FIG. 3 shows patient interface device 40 disposed in a first position, such as when the device is first placed on or near the face of the patient and generally secured thereto by a headgear 43 (shown in dashed line). In such position, an upper portion 44 (which is adapted to sealingly engage about the patient's nares) of cushion 42 is disposed a distance (not labeled) from the nose of the patient while a lower portion 50 (the portion adapted to sealingly engage about the mouth of the patient) is disposed about the mouth of the patient. FIG. 4 shows patient interface device 40 disposed in a second position, such as when the corresponding portions 44 and 50 of cushion 42 have been adjusted to sealingly engage about the nares and mouth of the patient.

Patient interface device 40 includes a port 45, or other suitable member, for receiving a flow of pressurized breathing gas from a conduit (not shown) coupled thereto.

In order to assist in adjusting the relative positioning of upper and lower portions 44 and 50 of cushion 42, patient interface device 40 may be provided with a first frame member 46 coupled to upper portion 44 of cushion 42 and a second frame member 48 coupled to lower portion 50 of cushion 42. The relative positioning of first and second frame members 46 and 48 may be selectively fixed or adjusted through the use of a mechanism 52 disposed in engagement with both of first and second frame members 46 and 48. Mechanism 52 may be formed as a screw-like or other suitable adjustment mechanism which allows for generally precise positioning of first and second frame member 46 and 48 with respect to each other. Such an arrangement would generally require the patient or medical caregiver to adjust the device for proper fitment on the face of the patient. Alternately, mechanism 52 may be formed as a spring-like member which acts to bias first and second frame members 46 and 48 in a direction generally away from each other, and thus generally bias the corresponding portions of cushion 42 toward the mouth and nose of the patient. Such an arrangement generally provides for a self adjusting device which generally does not require further adjustment by the patient or medical caregiver.

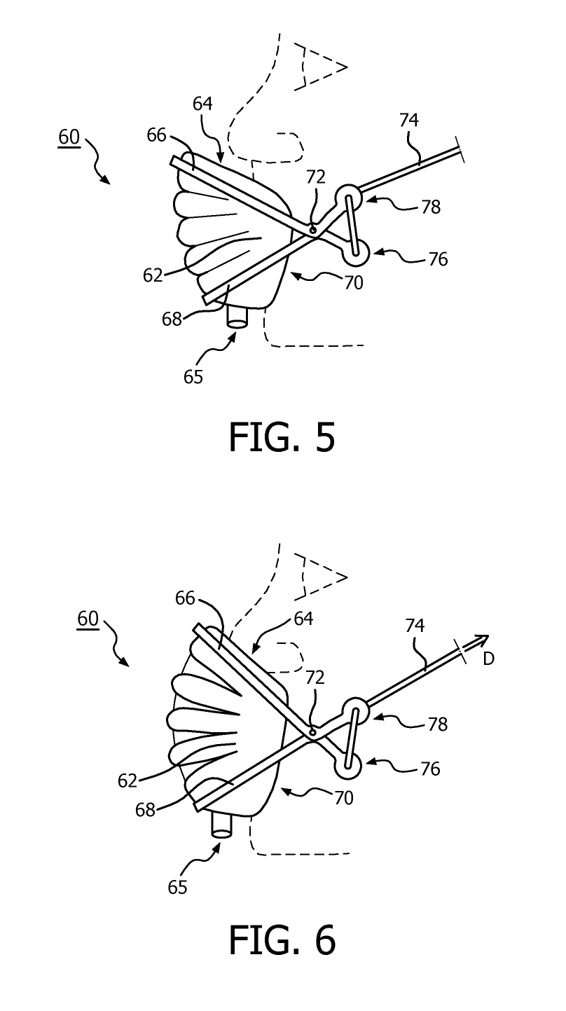

FIGS. 5 and 6 show side views of yet another example embodiment of a patient interface device 60 which functions in a similar manner as patient interface device 40 previously discussed. Patient interface device 60 includes a cushion 62 similar to cushions 22 and 42 (previously discussed) disposed on the face of a patient (shown in dashed lines). FIG. 5 shows patient interface device 60 disposed in a first position, such as when the device is first placed on or near the face of the patient. In such position, the upper portion 64 (which is adapted to sealingly engage about the patient's nares) of cushion 62 is disposed a distance (not labeled) from the nose of the patient while a lower portion 70 (the portion adapted to sealingly engage about the mouth of the patient) is disposed about the mouth of the patient. FIG. 6 shows patient interface device 60 disposed in a second position, such as when the corresponding portions 64 and 70 of cushion 62 have been adjusted to sealingly engage about the nares and mouth of the patient.

Patient interface device 60 includes a port 65, or other suitable member, for receiving a flow of pressurized breathing gas from a conduit (not shown) coupled thereto.

In order to assist in adjusting the relative positioning of upper and lower portions 64 and 70 of cushion 62, patient interface device 60 is provided with a first frame member 66 coupled to upper portion 64 of cushion 62 and a second frame member 68 coupled to lower portion 70 of cushion 62. First and second frame members 66 and 68 are pivotally coupled to each other at a pivot point 72 such that first and second frame members 66 and 68 interact in a reverse-scissor action as bringing either set of ends of the first and second frame members 66 and 68 together results in the opposite set of ends moving away from each other.

A cord 74 fixedly coupled to a rear portion 76 of second frame member 68 and slidingly engaged with a rear portion 78 of first frame member 66 provides for upper and lower portions 64 and 70 of cushion 62 to be biased generally away from each other when a force is applied to cord 74 in a direction away from frame members 66 and 68, such as in a direction D as shown in FIG. 6. In order to fix the relative positioning of frame members 66 and 68, a locking mechanism which selectively fixes cord 74 with respect to first frame member 66 may be provided on one or both of first frame member 66 or cord 74. In order to provide a level of automatic adjustability, cord 74 may be formed from an elastic material which allows for a selectable level of movement of frame members 66 and 68 with respect to each other due to stretching of cord 74.

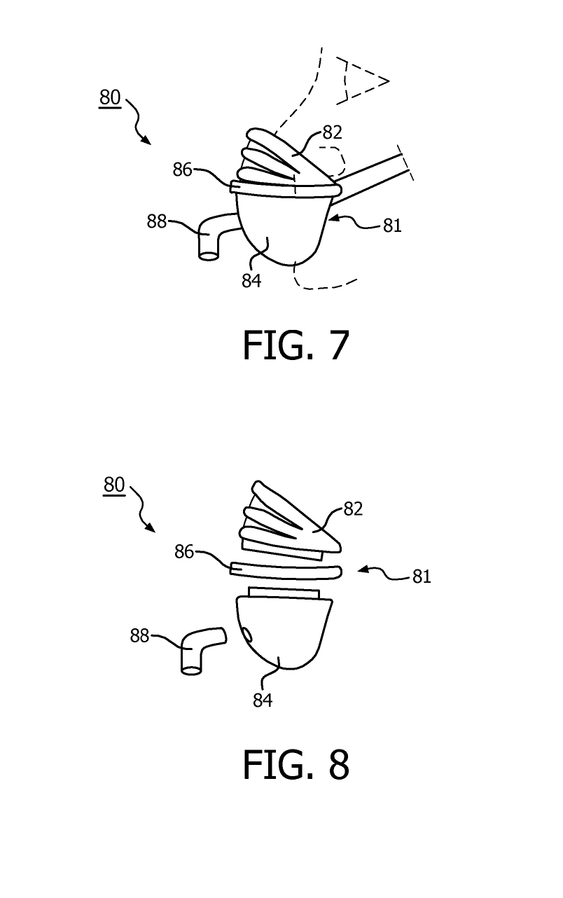

FIG. 7 shows a side view of a further example patient interface device 80 in accordance with the principles of the present invention disposed on the face of a patient (shown in dashed line) and FIG. 8 shows an exploded side view of patient interface device 80. Patient interface device 80 includes a cushion 81 having a first portion 82 adapted to sealingly engage about the nares of a patient, a second portion 84 adapted to sealingly engage about the mouth of a patient, and a third portion 86 disposed between, and selectively coupled to, first and second portion 82 and 84. Such selective coupling of first and second portions 82 and 84 to third portion 86 may be accomplished via a snap fit or other suitable sealable coupling without varying from the scope of the present invention. In the arrangement illustrated in FIGS. 7 and 8, the sizing of one or more of portions 82, 84 or 86 may be selectively varied to provide optimum fitment and comfort to a user. As an example, different third portions 86 of varied height may be employed which vary the spacing between first and second portion 82 and 82, thus allowing for patient interface device 80 to be fitted to patients of varying facial dimensions. Additionally, although shown generally as a nasal mask, first portion 82 may also be arranged in other forms (e.g., without limitation, a pillows mask, a cradle mask) without varying from the scope of the present invention

In order to facilitate the reception or exhalation of a flow of pressurized breathing gas, one or more of first, second or third portion 82, 84 or 86 may be provided with an elbow or port. In the example embodiment shown in FIGS. 7 and 8, second portion 84 is provided with an elbow 88 for such purposes. Such arrangement is provided for example purposes only and is not intended to be limiting upon the scope of the present invention.

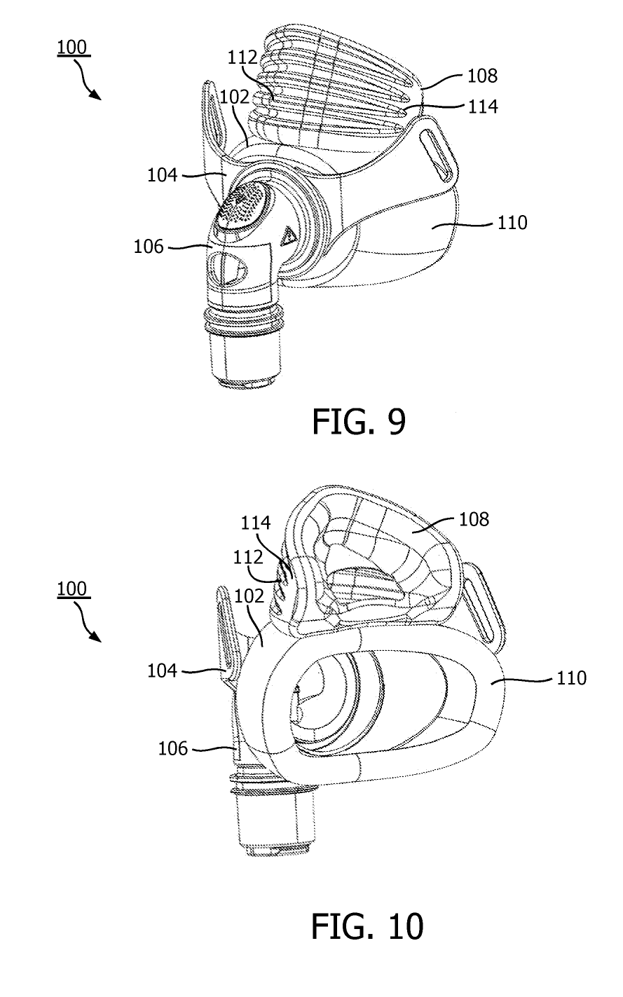

FIGS. 9 and 10 show isometric views of yet a further example patient interface device 100 in accordance with the principles of the present invention. Patient interface device 100, similar to patient interface device 14 previously described in conjunction with FIGS. 1 and 2, includes a cushion 102 and a generally rigid frame 104 coupled thereto. An opening in frame 104, to which elbow 106 is coupled, allows a flow of breathing gas to be communicated to interior spaces (not numbered) defined by cushion 102 and then to the airway of the patient.

Referring to FIG. 10, cushion 102 includes a first portion 108 adapted to sealingly engage about the nares of the patient. As shown in the illustrated embodiment, first portion 108 may be formed as a nasal mask, however, it is to be appreciated that first portion 108 may also be arranged in other forms (e.g., without limitations, a pillows mask, a cradle mask) without varying from the scope of the present invention.

Continuing to refer to FIG. 10, cushion 102 also includes a second portion 110 adapted to sealingly engage about the mouth of the patient. Cushion 110 further includes a third portion 112 is configured to allow for the relative positioning of first portion 108 and second portion 110 to be generally selectively adjusted so as to provide for optimum fitting of cushion 102, and thus patient interface device 100, to the face of the patient. By allowing relative motion between the nasal and oral portions (portions 108 and 110) of the mask, a better seal and improved comfort for the patient is achieved.

Unlike cushion 22 of patient interface device 14 in which first, second and third portions 28, 30 and 32 were integrally formed, only first and third portions 108 and 112 of cushion 102 are integrally formed as a single member 114 which is then coupled to second portion 110 via adhesive or other suitable coupling. Such arrangement allows for the use of different materials in different portions of cushion 102 as desired for particular applications.

From the above description it is to be appreciated that embodiments of the present invention provide for a cushion in which the relationship of the nasal and oral sealing portions of a cushion or cushion assembly may be adjustably provided. As a result, such cushion, and thus the patient interface with which it is employed, may be better fit to the face of a patient. By providing such improved fitment, a cushion and or patient interface device employing such cushion constructed according to the present invention provides for a more comfortable and better sealing fit than other cushions presently being used.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A cushion for use in a patient interface device, the cushion comprising:
a first portion adapted to sealingly engage around the nares of a patient;
a second portion adapted to sealingly engage around the mouth of the patient; and
a third portion configured to provide for the relative positioning of the first portion and the second portion to be selectively adjusted.

2. The cushion of claim 1, wherein the third portion is formed as a part of the second portion.

3. The cushion of claim 1, wherein the third portion is formed as a part of the first portion.

4. The cushion of claim 1, wherein the first, second and third portions are portions of a single integral member.

5. The cushion of claim 1, wherein the first and second portions are formed as separate members which have been coupled together.

6. The cushion of claim 1, wherein the third portion is disposed between the first portion and the second portion.

7. The cushion of claim 1, wherein the first portion comprises one of a nasal mask, a pillows mask, or a cradle mask.

8. The cushion of claim 1, wherein the third portion comprises a bellows-like structure.

9. The cushion of claim 1, wherein the first, second and third portions are separate members selectively coupled together.

10. A patient interface device comprising a cushion having:
a first portion adapted to sealingly engage around the nares of a patient; and
a second portion adapted to sealingly engage around the mouth of the patient, wherein at least one of the first portion or the second portion is movable from a first position in which the first portion and the second portion are disposed relative to each other and a second position in which the first portion and the second portion are disposed in a different relative position with respect to each other.

11. The patient interface device of claim 10, further comprising an adjustment mechanism coupled to the first portion and the second portion, the adjustment mechanism being adapted to selectively fix the first portion and the second portion in at least one of the first position or the second position.

12. The patient interface device of claim 11, wherein the adjustment mechanism comprises:
a first frame member coupled to the first portion of the cushion;
a second frame member coupled to the second portion of the cushion; and
a mechanism engaged with both the first frame member and the second frame member, the mechanism being adapted to selectively fix the relative positioning of the first frame member and the second frame member with respect to each other.

13. The patient interface device of claim 11, wherein the mechanism comprises one of a screw-like member or a cord member.

14. A method of fitting a patient interface device having a cushion including a first portion adapted to sealingly engage around the nares of a patient and a second portion adapted to sealingly engage around the mouth of the patient, the method comprising:
disposing the patient interface device on the head of the patient such that one of the first portion and the second portion is sealingly engaged about a corresponding one of the nares or mouth of the patient and the other of the first portion and the second portion is spaced a distance from the other one of the nares or mouth of the patient; and
adjusting the relative positioning of the first portion and the second portion to bring the other one of the first portion and the second portion into sealing engagement about the other one of the nares or mouth of the patient.

15. The method of claim 14, wherein adjusting the relative positioning of the first portion and the second portion comprises selectively fixing the relative positioning of the first and second portions with respect to each other.
